(19) United States
(12) Patent Application Publication
(10) Pub. No.: US 2009/0318884 A1
(43) Pub. Date: Dec. 24, 2009

(54) ABSORBENT STRUCTURES WITH IMMOBILIZED ABSORBENT MATERIAL

(76) Inventors: Axel Meyer, Frankfurt (DE); Robin Lynn McKiernan, Mason, OH (US); Jean-Phillippe Marie Autran, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/477,947

(22) Filed: Jun. 4, 2009

Related U.S. Application Data
(60) Provisional application No. 61/074,318, filed on Jun. 20, 2008.

(51) Int. Cl. A61F 13/53; A61L 15/58

(52) U.S. Cl. 604/366; 604/372

(57) ABSTRACT

The present disclosure relates to improved absorbent structures containing water-absorbing material that is immobilized with a matrix material and by use of a coating or partial coating of an immobilization enhancing component on the water-absorbing polymeric particles. This also relates to the use of an immobilization enhancing component in an absorbent structure including an absorbent component that includes a water-absorbing material and a matrix material. The absorbent structure is suitable, for example, as absorbent core in an adult incontinence article, infant (baby, toddler) diapers, including training or pull-on pants, and feminine hygiene article, such as sanitary napkins.

### CROSS REFERENCE TO RELATED APPLICATIONS

This application claims the benefit of U.S. Provisional Application No. 61/074,318, filed on Jun. 20, 2008, which is incorporated herein by reference.

### FIELD OF THE INVENTION

The present disclosure relates to improved absorbent structures containing water-absorbing material that is immobilized with a matrix material and an immobilization enhancing component. This also relates to the use of an immobilization enhancing component in an absorbent structure including an absorbent component that includes a water-absorbing material and a matrix material. The absorbent structure is suitable, for example, as absorbent core in an adult incontinence article, infant (e.g. baby, toddler) diapers, including training pants, and feminine hygiene article, such as sanitary napkins.

### BACKGROUND OF THE INVENTION

An important component of disposable absorbent articles such as diapers is an absorbent core (structure) including water-absorbing polymeric material, typically hydrogel-forming water-absorbing polymeric material, also referred to as absorbent gelling material, AGM, or super-absorbent polymer, SAP. This polymer material ensures that large amounts of bodily fluids, e.g. urine, can be absorbed by the article during its use and locked away, thus providing low rewet and good skin dryness. Especially useful water-absorbing polymeric material or SAP are often made by initially polymerizing unsaturated carboxylic acids or derivatives thereof, such as acrylic acid, alkali metal (e.g., sodium and/or potassium) or ammonium salts of acrylic acid, alkyl acrylates, and the like.

Traditionally, these water-absorbing polymers are incorporated into absorbent structures with cellulose or cellulosic fibres to provide an absorbent structure wherein the water-absorbing polymers can swell and absorb large quantities of urine with a reduced risk of gel-blocking and/or to ensure the right gelbed porosity or permeability, and also to ensure the absorbent structure is stable in use or during transport.

In recent years, the focus has been to make thinner absorbent structures. Hereto, it has been proposed to reduce or eliminate these cellulose fibres from the absorbent structures. However, the absorbent structure looses part of its mechanical stability in use without the presence of cellulose fibres, and the water-absorbing structure may suffer from gel-blocking.

It has thus been proposed to use other matrix materials, in smaller quantities or volumes, such as fibrous adhesives, to provide absorbent structures that have the required permeability/porosity, and reduced gel-blocking, and that form a stable structure in use or transport.

However, these matrix materials still provide extra volume to the absorbent structure and furthermore, they may be expensive. Therefore, there is a need to find further ways or alternative ways to make absorbent structures with low levels of matrix materials.

Also, the inventors found that some of these known matrix materials, such as some of the fibrous thermoplastic adhesive materials, may have a negative effect on the performance of the water-absorbing polymers. It is believed that this may be due to the interaction of these matrix materials with the surface of the water-absorbing polymers, and/or other diaper components (such as nonwoven materials), rendering their surface more hydrophobic, and thereby reducing the affinity of these surfaces with hydrophilic materials like urine, and thus reducing the absorbency of urine by the water-absorbing polymers. The inventors thus found that, on one hand, the use of matrix materials is essential to the performance of the water-absorbing structures, but that on the other hand it may be desirable to reduce the amount of matrix material used in absorbent structures.

They have now found a way to allow the reduction of the amount of matrix materials without negatively impacting the stability and immobilization and performance of the water-absorbing polymers (and the water-absorbent structure) by use of an additional immobilization enhancing material, applied to the water-absorbing material.

### SUMMARY OF THE INVENTION

The present disclosure relates to the use in an absorbent structure, that includes a substrate material and an absorbent component, the absorbent component including water-absorbing polymeric particles and a matrix material, of an immobilization enhancing component, which is applied to the surface of the water-absorbing polymeric particles, and believed to form an area of attachment for the matrix material, to aid immobilization of the water-absorbing polymeric particles by the matrix material in the absorbent structure.

The matrix material may include a thermoplastic material, or an adhesive material, or a thermoplastic adhesive material, and in one embodiment it includes a fibrous thermoplastic and/or adhesive material. It may have a viscosity of between 800 and 3000 mPa·s, or from 1000 mPa·s or 1200 mPa·s or from 1600 mPa·s to 2800 mPa·s or 2500 mPa·s, all at 175° C., as measurable by ASTM D3236-88, using spindle 27, 20 rpm, 20 minutes preheating at the temperature, and stirring for 10 min. Alternatively, it may have a viscosity of between 1500 and 4500 mPa·s, or from 2000 to 4000 mPa·s, or from 2300 to 3700 mPa·s, at 149° C. and as measurable by ASTM D3236-88, using spindle 27, 50 rpm, 30 minutes stirring. Alternatively, mixtures of such adhesive materials may be used, one or more having the first viscosity parameters above, and one or more having the second viscosity parameters above

In one embodiment, the matrix material is an aliphatic material (polymeric material) and the immobilization enhancing component is an aliphatic component (e.g. polymeric component). The matrix material may include polyolefin, polyester, polyether, polyamide, poly urea, and/or polyurethane units.

The immobilization enhancing component may include a polymeric material that has polyolefin, polyester, polyether, polyamide poly urea, and/or polyurethane units. This may be a co-polymeric material and/or a block co-polymeric material. It may include polyurethane and/or polyurea units.

In one embodiment the immobilization enhancing component is applied to the surface of the absorbent polymeric particles at a temperature of, or with subsequent heating at a temperature of above 150° C., above 180° C. or above 200° C. This heat applying step may be performed for, for example, at least 1 min., or at least 5 min., or at least 10 min., or at least 20 min.

The present disclosure also relates to an absorbent structure including a substrate material and an absorbent component, the absorbent component including a) water-absorbing polymeric particles, the particles having a coating or partial coating of an immobilization enhancing component; and b) a matrix material, whereby the weight ratio of the component a) to b) is from 10:1 to 200:1, (or for example from 10:1 to 100:1, or from 15:1 or 20:1 to 100:1 or from 35:1 to 100:1), and the immobilization enhancing component is present at a weight level (by weight of the absorbent component) of from 0.01% to 2%, and up to 1% by weight, and whereby the water-absorbing polymeric particles with the coating or partial coating may have a mean particle size between 200 and 850 microns, or between 200 and 700 microns, or between 200 and 600 microns, and/or having less than 10% by weight of particles with a particle size of less than 100 microns or less than 150 microns.

The present disclosure also relates to the process to make an absorbent structure including a substrate material and an absorbent component, the absorbent component including a) water-absorbing polymeric particles, the particles having a coating or partial coating of an immobilization enhancing component; and b) a matrix material, whereby the immobilization enhancing agent is applied to the polymeric particles, optionally under application of heat, for example at a temperature of at least 150° C., or at least 200° C., optionally followed by cooling; and then addition of the matrix material (for example at a temperature below 150° C.), whereby the weight ratio of the component a) to b) may be from 10:1 to 100:1 and the immobilization enhancing component is present at a weight level (by weight of the absorbent component) of from 0.01, or from 0.05% to 2%, up to 1% by weight, and whereby the water-absorbing polymeric particles with the coating or partial coating may have a mean particle size of from 200 to 800 microns and/or a particle size distribution such that less than 10% by weight of the particles has a particle size of below 150 microns.

In one embodiment the immobilization enhancing component may be applied to the surface of the water-absorbing polymeric particles at a temperature above the processing temperature of the matrix component, and/or at a temperature that is above the highest Tg of the thermoplastic polymeric component (adhesive) of the matrix material. In another embodiment, the immobilization enhancing component includes a plasticizer, and may be applied at any temperature to the water-absorbing polymeric particles.

In another embodiment, the immobilization enhancing component is applied as a solution to the surface of the water-absorbing polymeric particles, at any temperature.

In one embodiment, the absorbent structure herein may be free of fibrous absorbent cellulosic or cellulose material.

### DETAILED DESCRIPTION OF THE INVENTION

“Absorbent structure” refers to any three dimensional structure, including water-absorbing material, useful to absorb and retain liquids, such as urine, menses or blood. As described herein the absorbent structure may be absorbent article, as defined below, or the absorbent structure may be an absorbent component of such an article, e.g. an absorbent core. If the absorbent structure is part of a (disposable) absorbent article, then the absorbent structure may be that part of an absorbent article which serves to acquire and/or store bodily fluids, the absorbent structure may be the absorbent core, and/or the storage layer of and/or the acquisition layer of the article. The absorbent core of an absorbent article typically includes two or more layers, for example a storage layer and an acquisition layer, and then, one or more or all of the layers may be the absorbent structure as described herein.

“Absorbent article” refers to devices that absorb and retain liquids (such as blood, menses and urine), and more specifically, refers to devices that are placed against or in proximity to the body of the wearer to absorb and contain the various exudates discharged from the body. Absorbent articles include but are not limited to diapers (including diapers with fasteners, training pants, adult incontinence diapers), adult incontinence briefs, diaper holders and liners, feminine hygiene articles, including sanitary napkins and the like.

“Diaper” refers to an absorbent article generally worn by infants and incontinent persons about the lower torso; infant diaper refers to baby and toddler diapers, including training pants, worn about the lower torso.

“Disposable” is used herein to describe articles that are generally not intended to be laundered or otherwise restored or reused (i.e., they are intended to be discarded after a single use and, may be recycled, composted or otherwise disposed of in an environmentally compatible manner).

The absorbent structure herein includes a substrate material and a water-absorbing component, which includes water-absorbing polymeric particles and a matrix material, and an immobilization enhancing material, applied to the surface of the water-absorbing polymeric particles, e.g. as a coating or partial coating.

The absorbent structure may typically be a layer with a Z-direction thickness, and a width (X-direction) and length (Y-direction), and having for example a surface area on X-Y direction of at least 1 cm2, at least 5 cm2, and in some absorbent articles herein, at least 20 cm2, and in some embodiments at least 100 cm2, and/or having for example a volume of at least 1 cm3, or at least 5 cm3 or at least 10 cm3 (when laid out flat, under normal atmospheric pressure, conditioned for 24 hrs at 20° C., 50% relative humidity).

The substrate of the structure herein may be any material, e.g. layer or sheet, capable to hold, or support or contain water-absorbing polymers. Typically, it is a web or sheet material, such as a foam, film, woven web and/or nonwoven web, as known in the art. The substrate may include spunbond, meltblown and/or carded nonwovens. A material may be a so-called SMS material or SMMS material, including a spunbond layer, one or two, respectively, melt-blown layers and a further spunbond layer. Some may be permanently hydrophilic nonwovens, and/or nonwovens with hydrophilic coatings. The substrate material may enclose the absorbent component herein. The substrate material may include a top layer and the bottom layer, which may be made of a unitary material, in which case this material is folded to form a top and bottom layer, or it may be made of two or more separate sheets or webs. The substrate is typically made of one or more sheets or layers that are bonded together to enclose the water-absorbing component therein, e.g. by adhesive bonding and/or heat bonding.

Nonwoven materials may be provided from synthetic fibres, such as polyethylene, PET and polypropylene. As the polymers used for nonwoven production may inherently be hydrophobic, they may be coated with hydrophilic coatings, e.g., coated with nanoparticles, as known in the art.

Some nonwoven materials and absorbent structures using such materials are described in, for example, co-pending applications US 2004/0162536, EP1403419-A, WO2002/0192366, EP 147028 1-A and EP 1470282-A.

Absorbent structures that may be used in absorbent articles herein include a layer of a substrate material and thereon a layer that is a water-absorbent component layer (which may be discontinuous and/or profiled in Z, X or Y direction), including water-absorbing polymeric particles and a matrix material, which may be present in the form of separate layers, and/or they may be mixed. The immobilization enhancing component, described herein, is present on at least part of the surface of the water-absorbing polymeric particles such that it is also in contact with the matrix material.

Thereby, the matrix material can provide a matrix, e.g. cavities or network, to hold the water-absorbing material and the immobilization enhancing material aids thereby to immobilize the water-absorbing material, e.g. in the cavities or network.

Absorbent structures can for example be made as follows:
a) providing one or more substrate materials (together forming the substrate, as referred to herein), e.g. that can serve as a wrapping or partial wrapping material;
b) providing a water-absorbing material including water-absorbing polymeric particles that include an immobilization enhancing material, e.g. as coating or partial coating on the surface of the particles;
c) providing a matrix material;
   and then forming a water-absorbent component by either:
d) depositing the matrix material on the substrate material and then the water-absorbing material onto the matrix material; and/or
e) depositing the water-absorbent material on the substrate material and then the matrix material onto the water-absorbent material; and/or
f) mixing the matrix material and water-absorbent material and then depositing the mixture on the substrate material; and then:
g) enclosing the resulting component with the substrate material(s) and typically sealing the substrate material; or
h) repeating steps a) to f) to obtain two or more absorbent components which are then combined to form the final absorbent component, and then applying step g) above, to obtain the absorbent structure, typically by ensuring the substrate materials of each component form one of the outer surfaces of the absorbent structure.

Optionally, the water-absorbent material and/or matrix material and/or mixture thereof may be applied in a pattern with varying dimensions, e.g. thickness, width or length, and/or a pattern, so that the absorbent structure includes at least one zone which is substantially free of water-absorbing material, and at least one or at least two zones including water-absorbing material (such that openings may be formed between the separate zones with water-absorbing material).

As described herein, the immobilization enhancing component may be added to the water-absorbing polymeric particles by any method, suitable to form a partial coating or coating of the component on the particles, and it may for example be done during the surface-crosslinking step or after the surface-crosslinking step.

As described above, the water-absorbent structure includes a water-absorbent component that includes a matrix material and water-absorbent material, which includes water-absorbent polymeric particles. The water-absorbent polymeric particles include, typically on the particle surface as a partial coating or complete coating, e.g. uniform coating, an immobilization enhancing component. The immobilization enhancing component may be present at a level of less than 5% by weight of the absorbent component, at a level of from 0.01% to 5%, or from 0.01% or from 0.05% to 2% or to 1% or to 0.8 % by weight (of the absorbent component). The weight ratio of the water-absorbent polymeric particles, or the water-absorbent polymeric particles including the coating or partial coating of the immobilization enhancing component, to the matrix material may be from 10:1 to 200:1, or any of the ratio's described above. The absorbent component may include the matrix material, water-absorbent polymeric particles and immobilization enhancing component. The water-absorbent polymeric particles may be present at a level of at least 89% or at least 90%, by weight of the water-absorbent component, or even at least 92% or at least 93% or at least 94%, by weight of the water-absorbent component. The matrix material may be present at a level of from 1% to 10% by weight of the absorbent component, or from 2% or 3% to 7% or to 5% by weight.

Articles herein may have a topsheet and a backsheet, which each has a front region, back region and crotch region, positioned therein between. If the absorbent structure herein is an absorbent core, then it is typically positioned in between the topsheet and backsheet of an absorbent article. Backsheets may be vapor pervious but liquid impervious. The topsheet materials may be at least partially hydrophilic. So-called apertured topsheets may also be used. Topsheets with one or more (large) openings may also be used. The topsheet may also include a skin care composition, e.g., a lotion.

Diapers according to the present disclosure may achieve a relatively narrow crotch width, which increases the wearing comfort. One example article achieves a crotch width of less than 100 mm, 90 mm, 80 mm, 70 mm, 60 mm or even less than 50 mm, as measured along a transversal line which is positioned at equal distance to the front edge and the rear edge of the article, or at the point with the narrowest width. Hence, an absorbent structure may have a crotch width as measured along a transversal line which is positioned at equal distance to the front edge and the rear edge of the core which is of less than 100 mm, 90 mm, 80 mm, 70 mm, 60 mm or even less than 50 mm. It has been found that for most absorbent articles the liquid discharge occurs predominately in the front half.

Diapers herein may have a front waist band and a back waist band, whereby the front waist band and back waist band each have a first end portion and a second end portion and a middle portion located between the end portions, and whereby the end portions each include a fastening system, to fasten the front waist band to the rear waist band or whereby the end portions may be connected to one another, and whereby the middle portion of the back waist band and/or the back region of the backsheet and/or the crotch region of the backsheet includes a landing member, wherein the landing member may include second engaging elements selected from loops, hooks, slots, slits, buttons, magnets. Some may be hooks, adhesive or cohesive second engaging elements. It may be that the engaging elements on the article, or diaper are provided with a means to ensure they are only engagable at certain moments, for example, they may be covered by a removable tab, which is removed when the engaging elements are to be engaged and may be reclosed when engagement is no longer needed, as described above.

Diapers (including training pants) herein may have one or more sets of leg cuffs and/or barrier leg cuffs, as known in the art. It may also be that diaper has a secondary topsheet, in contact with the skin and may be overlaying a primary topsheet, as for example described above), the secondary topsheet having an elongated slit opening, possibly with elastication means along the length thereof, where through waste material can pass into a void space above the absorbent structure, and which ensures the waste material is isolated in this void space, away from the wearer's skin.

In one embodiment herein, the diaper is an infant (baby, toddler) diaper, including training pants, with an absorbent structure, e.g. absorbent core, or absorbent layer, which includes an absorbent component, the absorbent component including a) the water-absorbing polymeric particles, the particles having a coating or partial coating of the immobilization enhancing component; and b) a matrix material, that may be present at a level of from 1% to 10% by weight, from 4% to 10% by weight, or from 4 to 8% by weight, whereby the weight ratio of the component a) to b) is from 10:1 to 100:1 and the immobilization enhancing component is present at a weight level (by weight of the absorbent component) of from 0.01% to 2%, and may be up to 1%, or from 0.05% to 0.5% by weight, and the absorbent structure including less than 10%, or less than 5%, or even less than 1% by weight or no fibrous absorbent cellulose material. Then, the use of the immobilization enhancing component may provide an improvement of immobilization of the water-absorbing polymeric particles of at least 20%, or at least 40%, or at least 50% or even at least 60%, as determined by the Wet Immobilization Test.

The water-absorbing polymeric particles, with the coating or partial coating, may have a mean particle size of from 200 to 800 microns and/or a particle size distribution such that less than 10% by weight of the particles has a particle size of below 150 microns.

### Matrix Material

The absorbent structure may include an absorbent component that includes a matrix material. The matrix material may be an adhesive matrix material and/or thermoplastic matrix material and/or a fibrous matrix material, a fibrous thermoplastic adhesive matrix material. In one embodiment herein, the matrix materials do not absorb urine or water.

In one embodiment, the matrix material is fibrous. It may be an adhesive material. It may be a thermoplastic fibrous adhesive material, so that the matrix material the in the form of fibres to provide the required matrix for the absorbent polymeric particles. The matrix material may include an ethylene vinyl acetate derivative, styrenic block copolymer derivative and/or a polyolefin derivative, as further describe below.

The matrix material may be or include or be a thermoplastic component, e.g. polymeric component, including a single thermoplastic polymer or a blend of thermoplastic polymers, for example having a softening point, as determined by the ASTM Method D-36-95 “Ring and Ball”, in the range between 50° C. and 300° C., or alternatively the matrix material may include or be a thermoplastic (hot melt) adhesive including at least one thermoplastic polymer in combination with additives, including other thermoplastic compounds, including for example tackifying resins, plasticizers and/or additives such as antioxidants and/or stabilisers. In one embodiment the thermoplastic polymer or polymers are aliphatic.

The thermoplastic polymer included by the matrix material may have a weight average molecular weight (Mw) of more than 10,000 g/mole; it may have a glass transition temperature (Tg) usually below room temperature (20° C.), as determined by ASTM E1356-03. It may have two or more Tg's, and then may have at least one Tg, but it may be that not all Tg's, are below room temperature. A wide variety of thermoplastic polymers are suitable for use. Such thermoplastic polymers may be water insensitive.

Some are polymers, copolymers or block copolymers with a polyolefin, polyether, polyester and/or polyamide units.

Example polymers include styrenic block copolymers (SBC), ethylene vinyl acetate polymers (EVA), or amorphous poly-alpha olefin (APAO).

Exemplary polymers are styrenic block copolymers including A-B-A triblock structures, A-B diblock structures and (A-B)n radial block copolymer structures wherein the A blocks are non-elastomeric polymer blocks, typically including polystyrene, and the B blocks are unsaturated conjugated diene or hydrogenated versions of such. The B block is typically isoprene, butadiene, ethylene/butylene (hydrogenated butadiene), ethylene/propylene (hydrogenated isoprene), and mixtures thereof. It may be that the thermoplastic polymer includes a styrene-isoprene-styrene (SIS), and/or a styrene-butadiene-styrene (SBS) and/or styrene-ethylene/butylene-styrene (SEBS), or SIS. The triblock may for example include about 14-22 weight % styrene for SIS copolymers and above 25 weight % styrene for SBS copolymers. Triblock can also contain 0-50 weight % of diblock.

One example matrix material may include EVA polymers. EVA polymers are copolymers of ethylene and vinyl acetate. The vinyl acetate is generally in the range of 15-40 weight %.

Other suitable thermoplastic polymers that may be employed are metallocene polyolefins, which are ethylene polymers prepared using single-site or metallocene catalysts. Therein, at least one comonomer can be polymerized with ethylene to make a copolymer, terpolymer or higher order polymer. Also applicable are amorphous polyolefins or amorphous polyalphaolefins (APAO) which are homopolymers, copolymers or terpolymers of C2 to C8 alphaolefins, for example of propene-ethylene, propene-butene, propene-hexene, or terpolymers of propene-butene-ethylene made by a Ziegler-Natta polymerization.

A tackifier or tackifying resin may be present, Such a tackifying agents may have a weight average Mw below 5,000 g/mole. The tackifying agent may have and a Tg above room temperature (20° C.). Typical concentrations of the tackifier or tackifying resin in the matrix material herein are in the range of 30-60%. The tackifier can be either from natural or synthetic sources. It may be a hydrogenated compound. Exemplary tackifiers include (natural) rosin (C20 mono-acid) and terpenes (C10) and (petroleum based) acyclic C5, aliphatic/aromatic C5/C9, dicyclopentadiene (2-C5 or cyclic C5), and aromatic pure monomer base and hydrogenated versions thereof. The natural tackifiers are mostly terpenes or rosin esters made by the esterification of natural rosin. The hydrocarbon or petroleum based tackifiers are low molecular weight polymers derived from monomers obtained from petroleum, coal, and plants. Typically the monomers are obtained from a naphtha cracker and then the monomers are usually cationically polymerized. The C5 and C9 resins are named for the petroleum stream they are obtained from and generally the C5 is more aliphatic in nature and the C9 is more aromatic in nature. The number roughly describes the number of carbon in the monomer unit, however it's not a hard rule and the C9 can easily contain monomers having 8-10 carbons in it. Useful tackifiers of C5 and C9 resins are described in the U.S. Pat. No. 6,310,154.

The matrix material may also include, in addition to a thermoplastic adhesive, a plasticizer, that is liquid or waxy at room temperature (20° C.), For suitable liquid (at 20° C.) plasticizers, the weight average molecular weight is typically low (<1,000 g/mole) and the glass transition is below room temperature. For a waxy plasticizer, the weight average molecular weight may be low (<2,000 g/mole) but the glass transition is above room temperature. It may be crystalline. Example plasticizers may include paraffinic and naphthenic oils, typically with low aromatic content.

A typical concentration of plasticizer is 0-50%, or 1% to 45% or 5% to 45%, or 10% to 40% by weight of the matrix material.

Furthermore, other additives may be added, such as stabilizers, chain terminators, UV protecting agents, antioxidants, and bacteriostats to help prevent thermal, oxidative, and bio-chemical degradation. Matrix materials including SBC may include end-block reinforcers, e.g. if the end use is for higher temperature applications. Examples of end-block reinforces include aromatic C9 and coumarone-indene compounds. The matrix material herein may also include inorganic particulate material, including pigments, e.g. zinc oxide, titanium dioxide, clay (hydrated aluminum silicate), silica (silicon dioxide, may be hydrated), talc (magnesium silicate) and whitening agents (calcium carbonate).

In one embodiment herein, it may be that the matrix material includes an adhesive that may include 30% to 70%, or up to 60% by weight of one or more thermoplastic polymers, such as SBS or EVA, and 10% to 50% by weight of a tackifier and may be 5% to 30%, 10-40%, or 20-30%, by weight % of plasticizer and optionally minor compounds like stabilizer.

The matrix material may be present in the forms of fibres, i.e. the matrix material may include a material, as described herein, and that is fiberized or fibrous.

The fibres may have an average thickness of 1-50 micrometer and an average length of 5 mm to 50 cm.

In the absorbent structures herein, the weight ratio of the water-absorbing polymers to the matrix material may be from 10:1 to 200:1, from 15:1 or from 20:1 to 100:1, or from 35:1 to 100:1.

### Water-Absorbing Materials

The water-absorbing material includes water-absorbing polymers that are typically particulate, herein referred to as polymeric particles; for example polymeric particles obtainable by polymerization of a monomer solution including
i) at least one alkylenically (ethylenically) unsaturated acid-functional monomer,
ii) at least one crosslinker,
iii) if appropriate one or more ethylenically and/or allylically unsaturated monomers copolymerizable with i) and
iv) if appropriate one or more water-soluble polymers onto which the monomers i), ii) and if appropriate iii) can be at least partially grafted,
   wherein the base polymer obtained thereby is dried, classified and—if appropriate—is subsequently treated with
v) at least one post-crosslinker
   before being dried and thermally post-crosslinked (i.e. surface crosslinked).

The immobilization enhancing component may be added during the post-crosslinking or surface crosslinking step, or just after this step but for example before the final drying step. So the polymeric particles obtained from step iv) may be surface cross-linked and coated with the immobilization enhancing component at about the same time.

Useful monomers i) include for example ethylenically unsaturated carboxylic acids, such as acrylic acid, methacrylic acid, maleic acid, fumaric acid, tricarboxy ethylene and itaconic acid, or derivatives thereof, such as acrylamide, methacrylamide, acrylic esters and methacrylic esters. Acrylic acid and methacrylic acid are example monomers.

The water-absorbing polymers that can be used may be crosslinked, i.e., the polymerization is carried out in the presence of compounds having two or more polymerizable groups which can be free-radically copolymerized into the polymer network. Useful crosslinkers ii) include for example ethylene glycol dimethacrylate, diethylene glycol diacrylate, allyl methacrylate, trimethylolpropane triacrylate, triallylamine, tetraallyloxyethane as described in EP-A 530 438, di- and triacrylates as described in EP-A 547 847, EP-A 559 476, EP-A 632 068, WO 93/21237, WO 03/104299, WO 03/104300, WO 03/104301 and in the DE-A 103 31 450, mixed acrylates which, as well as acrylate groups, include further ethylenically unsaturated groups, as described in DE-A 103 31 456 and DE-A 103 55 401, or crosslinker mixtures as described for example in DE-A 195 43 368, DE-A 196 46 484, WO 90/15830 and WO 02/32962.

Useful crosslinkers ii) include in particular N,N′-methylenebisacrylamide and N,N′-methylenebismethacrylamide, esters of unsaturated mono- or polycarboxylic acids of polyols, such as diacrylate or triacrylate, for example butanediol diacrylate, butanediol dimethacrylate, ethylene glycol diacrylate, ethylene glycol dimethacrylate and also trimethylolpropane triacrylate and allyl compounds, such as allyl (meth)acrylate, triallyl cyanurate, diallyl maleate, polyallyl esters, tetraallyloxyethane, triallylamine, tetraallylethylenediamine, allyl esters of phosphoric acid and also vinylphosphonic acid derivatives as described for example in EP-A 343 427. Useful crosslinkers ii) further include pentaerythritol diallyl ether, pentaerythritol triallyl ether, pentaerythritol tetraallyl ether, polyethylene glycol diallyl ether, ethylene glycol diallyl ether, glycerol diallyl ether, glycerol triallyl ether, polyallyl ethers based on sorbitol, and also ethoxylated variants thereof. The process may utilize di(meth)acrylates of polyethylene glycols, the polyethylene glycol used having a weight average molecular weight in the range from 300 g/mole to 1000 g/mole.

However, particularly advantageous crosslinkers ii) are di- and triacrylates of altogether 3- to 15-tuply ethoxylated glycerol, of altogether 3- to 15-tuply ethoxylated trimethylolpropane, especially di- and triacrylates of altogether 3-tuply ethoxylated glycerol or of altogether 3-tuply ethoxylated trimethylolpropane, of 3-tuply propoxylated glycerol, of 3-tuply propoxylated trimethylolpropane, and also of altogether 3-tuply mixedly ethoxylated or propoxylated glycerol, of altogether 3-tuply mixedly ethoxylated or propoxylated trimethylolpropane, of altogether 15-tuply ethoxylated glycerol, of altogether 15-tuply ethoxylated trimethylolpropane, of altogether at least 40-tuply ethoxylated glycerol and also of altogether at least 40-tuply ethoxylated trimethylolpropane. Where n-tuply ethoxylated means that n mols of ethylene oxide are reacted to one mole of the respective polyol with n being an integer number larger than 0.

Diacrylated, dimethacrylated, triacrylated or trimethacrylated multiply ethoxylated and/or propoxylated glycerols as described for example in WO 03/104301 may be used as crosslinkers. Di- and/or triacrylates of 3- to 10-tuply ethoxylated glycerol are particularly advantageous. Di- or triacrylates of 1- to 5-tuply ethoxylated and/or propoxylated glycerol may be used. The triacrylates of 3- to 5-tuply ethoxylated and/or propoxylated glycerol may be used. These are notable for particularly low residual levels in the water-absorbing polymer (typically below 10 ppm) and the aqueous extracts of water-absorbing polymers produced therewith have an almost unchanged surface tension compared with water at the same temperature (typically not less than 0.068 N/m).

Examples of ethylenically unsaturated monomers iii) which are copolymerizable with the monomers i) are acrylamide, methacrylamide, crotonamide, dimethylaminoethyl methacrylate, dimethylaminoethyl acrylate, dimethylaminopropyl acrylate, diethylaminopropyl acrylate, dimethylaminobutyl acrylate, dimethylaminoethyl methacrylate, diethylaminoethyl methacrylate, dimethylaminoneopentyl acrylate and dimethylaminoneopentyl methacrylate.

Useful water-soluble polymers iv) include polyvinyl alcohol, polyvinylpyrrolidone, starch, starch derivatives, polyglycols, polyacrylic acids, polyvinylamine or polyallylamine, partially hydrolysed polyvinylformamide or polyvinylacetamide, polyvinyl alcohol and starch.

Water-absorbing polymeric particles whose base polymer is lightly crosslinked may be used. The light degree of crosslinking is reflected in the high CRC value and also in the fraction of extractables.

Base polymers having a 16 h extractables fraction of not more than 20% by weight, and not more than 15% by weight, not more than 10% by weight and not more than 7% by weight may be used.

The preparation of a suitable base polymer and also further useful hydrophilic ethylenically unsaturated monomers i) are described in DE-A19941423, EP-A 686 650, WO 01/45758 and WO 03/14300.

The reaction may be carried out in a kneader as described for example in WO 01/38402, or on a belt reactor as described for example in EP-A-955 086.

The acid groups of the base polymers obtained may be 0-100 mol %, 25-100 mol %, 65-90mol % and 68-80 mol % neutralized, for which the customary neutralizing agents can be used, for example ammonia, or amines, such as ethanolamine, diethanolamine, triethanolamine or dimethylaminoethanolamine, alkali metal hydroxides, alkali metal oxides, alkali metal carbonates or alkali metal bicarbonates and also mixtures thereof, in which case sodium and potassium may be alkali metal salts or sodium hydroxide, sodium carbonate or sodium bicarbonate and also mixtures thereof. Typically, neutralization is achieved by admixing the neutralizing agent as an aqueous solution or as an aqueous dispersion or else as a molten or as a solid material.

The neutralized base polymer may then be dried with a belt, fluidized bed, tower dryer or drum dryer until the residual moisture content may be below 13% by weight, especially below 8% by weight and may be below 4% by weight, the water content being determined according to EDANA's recommended test method No. 430.2-02 “Moisture content” (EDANA=European Disposables and Nonwovens Association). The dried base polymer may thereafter be ground and sieved, useful grinding apparatus typically include roll mills, pin mills, hammer mills, jet mills or swing mills.

The water-absorbing polymers to be used may be post-crosslinked. Useful post-crosslinkers v) include compounds including two or more groups capable of forming covalent bonds with the carboxylate groups of the polymers. Useful compounds include for example alkoxysilyl compounds, polyaziridines, polyamines, polyamidoamines, di- or polyglycidyl compounds as described in EP-A 083 022, EP-A 543 303 and EP-A 937 736, polyhydric alcohols as described in DE-C 33 14 019. Useful post-crosslinkers v) are further the to include by DE-A 40 20 780 cyclic carbonates, by DE-A 198 07 502 2-oxazolidone and its derivatives, such as N-(2-hydroxyethyl)-2-oxazolidone, by DE-A 198 07 992 bis- and poly-2-oxazolidones, by DE-A 198 54 573 2-oxotetrahydro-1,3-oxazine and its derivatives, by DE-A 198 54 574 n-acyl-2-oxazolidones, by DE-A 102 04 937 cyclic ureas, by DE-A 103 34 584 bicyclic amide acetals, by EP-A 1 199 327 oxetanes and cyclic ureas and by WO 03/031482 morpholine-2,3-dione and its derivatives.

The at least one post-crosslinker v) is typically used in an amount of about 1.50 wt. % or less, may be not more than 0.50% by weight, may be not more than 0.30% by weight and may be in the range from 0.001% and 0.15% by weight, all percentages being based on the base polymer, as an aqueous solution. It is possible to use a single post-crosslinker v) from the above selection or any desired mixtures of various post-crosslinkers.

The aqueous post-crosslinking solution, as well as the at least one post-crosslinker v), can typically further include a cosolvent. Cosolvents which are technically highly useful are C1-C6-alcohols, such as methanol, ethanol, n-propanol, isopropanol, n-butanol, sec-butanol, tert-butanol or 2-methyl-1-propanol, C2-C5-diols, such as ethylene glycol, 1,2-propylene glycol, 1,3-propanediol or 1,4-butanediol, ketones, such as acetone, or carboxylic esters, such as ethyl acetate.

One particular embodiment does not utilize any cosolvent. The at least one post-crosslinker v) is then only employed as a solution in water, with or without an added deagglomerating aid. Deagglomerating aids are known to one skilled in the art and are described for example in DE-A-10 239 074 and also prior PCT application PCT/EP/05011073, which are each hereby expressly incorporated herein by reference. Deagglomerating aids may be surfactants such as ethoxylated and alkoxylated derivatives of 2-propylheptanol and also sorbitan monoesters. Example deagglomerating aids are Plantaren® (Cognis), Span® 20, Polysorbate® 20—also referred to as Tween® 20 or polyoxyethylene 20 sorbitan monolaurate, and polyethylene glycol 400 monostearate.

The water-absorbing polymeric particles may for example have a particle size distribution in the range from 45 μm to 4000 μm. Particle sizes used in the hygiene sector may be in range from 45 μm to 1000 μm, may be from 45-850 μm, and in one embodiment herein, the particle sizes are as specified above. The particle size can be obtained by standard methods, such as also described in U.S. Pat. No. 5,422,169.

Narrow particle size distributions may be those in which not less than 80% by weight of the particles, may be not less than 90% by weight of the particles and may be not less than 95% by weight of the particles are within the selected range; this fraction can be determined using the familiar sieve method of EDANA 420.2-02 “Particle Size Distribution”. Selectively, optical methods can be used as well, provided these are calibrated against the accepted sieve method of EDANA. Narrow particle size distributions may have a span of not more than 700 μm, may have not more than 600 μm, and may have less than 400 μm. Span here refers to the difference between the coarse sieve and the fine sieve which bound the distribution. The coarse sieve is not coarser than 850 μm and the fine sieve is not finer than 45 μm. Particle size ranges which are suitable for one embodiment herein are for example fractions of 150-600 μm (span: 450 μm), of 200-600 μm (span: 400 μm), of 300-600 μm (span: 300 μm), of 200-700 μm (span: 500 μm), 300-700 μm (span: 400 μm), of 400-800 μm (span: 400 μm).

Water-absorbing particles may contain less than 3 wt. %, may contain less than 1 wt. %, and may contain less than 0.5 wt. % particles with a particle size less than 150 μm.

### Immobilization Enhancing Component

The immobilization enhancing component herein includes a compound that can form a coating or partial coating on the water-absorbing particles and that have a chemical group that can provide an anchoring point for the matrix material. It may be that the immobilization enhancing component includes an aliphatic polymer, including copolymers and block (co) polymers.

Immobilization enhancing components herein may include, or consist of, polymeric compounds that have amide, amine, alcohol, polyhydroxy, ester or ether units, typically in the polymeric backbone, polyamide, polyamine, polyester or polyether units; it may be that such components that include polyurea units or blocks and/or polyurethane units or blocks, or that consist of polyurethane or polyurea. Also mixtures of such polymers may be used.

In one embodiment, the immobilization enhancing component or a polymeric material therein has a first solubility parameter and the matrix material or the thermoplastic adhesive compound thereof has a second solubility parameter, and the difference between the first and second solubility parameter is between 0 and 1 (cal/cm3)1/2 (as described in “Polymer Handbook, 3rd Edition, Ed. J Brandrup and E. H. Immergut, VII-522-526).

In one embodiment, the polymer or block copolymer may have polyurethane and/or polyurea blocks.

In one embodiment herein, the use of the immobilization enhancing component in a specific absorbent structure of a diaper, provides an improvement of immobilization of the water-absorbing polymeric particles of at least 20%, or at least 40%, as determined by the Wet Immobilization Test, as further described herein.

The immobilization enhancing component may be or include film-forming polymers, or elastomeric film-forming polymers, that can suitably provide a coating or partial coating on the water-absorbing polymeric particles. Film-forming means that the respective polymer can readily be made into a layer (e.g. coating) upon evaporation of the solvent in which it is dissolved or dispersed. Elastomeric means that the material will exhibit stress-induced deformation that is partially or completely reversed upon removal of the stress.

In one embodiment, the immobilization enhancing component is not water-soluble and/or not urine soluble. It may be that the immobilization enhancing component is not water-absorbing, e.g. less than 1 g/g.

The immobilization enhancing component is mixed with or applied to the water-absorbing polymers by any means, e.g. by any method suitable to coat or partially coat the water-absorbing polymers with the immobilization enhancing component. The immobilization enhancing component may be applied as a solid material, as a hotmelt, as a dispersion, including an aqueous dispersion, as a (non)-aqueous solution or as an organic solution. It may be that the immobilization enhancing polymer is mixed with and/or applied to the water-absorbing polymers as an organic solution or dispersion, or as an aqueous dispersion, using any suitable organic solvent for example acetone, isopropanol, tetrahydrofuran, methyl ethyl ketone, dimethyl sulfoxide, dimethylformamide, n-methylpyrrolidone, chloroform, ethanol, methanol or mixtures thereof.

Immobilization enhancing components may include polymers that have two or more glass transition temperatures (Tg) (determined by ASTM E1356-03). Ideally, the polymers used exhibit the phenomenon of phase separation, i.e., they contain two or more different blocks of low and high Tg side by side in the polymer (Thermoplastic Elastomers: A Comprehensive Review, eds. Legge, N. R., Holden, G., Schroeder, H. E., 1987, chapter 2). Phase-separating polymers, such as styrenic block copolymers, herein may include one or more phase-separating block copolymers, having a weight average molecular weight (Mw) of at least 5 kg/mol, or at least 10 kg/mol and higher.

In one embodiment such a block copolymer has at least a first polymerized homopolymer segment (block) and a second polymerized homopolymer segment (block), polymerized with one another, whereby the first (soft) segment may have a Tg1 of less than 28° C. or even less than 22° C., or even less than 2° C., and the second (hard) segment may have a Tg2 of at least 45° C., or of 50° C. or more, 60° C. or more or even 70° C. or more.

In another embodiment, such a block copolymer has at least a first polymerized polymer segment (block) and a second polymerized polymer segment (block), polymerized with one another, whereby the first (soft) segment may have a Tg1 of less than 28° C. or even less than 22° C., or even less than 2° C., and the second (hard) segment may have a Tg2 of at least 45° C., or of 50° C. or more, 60° C. or more or even 70° C. or more.

The weight average molecular weight of a first (soft) segment (with a Tg of less than 25° C.) may be at least 500 g/mol, at least 1000 g/mol or even at least 2000 g/mol, and may be less than 8000 g/mol, and may be less than 5000 g/mol.

However, the total of the first (soft) segments is typically 20% to 95% by weight of the total block copolymer, or even from 20% to 85% or more, from 30% to 75% or even from 40% to 70% by weight. Furthermore, when the total weight level of soft segments is more than 70%, an individual soft segment may have a weight average molecular weight of less than 5000 g/mol.

In one embodiment herein, the immobilization enhancing component includes one or more polyurethanes.

The polyurethane may be hydrophilic and in particular surface hydrophilic. The surface hydrophilicity may be determined by methods known to those skilled in the art. In one execution, the hydrophilic polyurethanes are materials that are wetted by the liquid that is to be absorbed (0.9% saline; urine). They may be characterized by a contact angle that is less than 90 degrees. Contact angles can for example be measured as set out in ASTM D 5725-99.

In one embodiment, the hydrophilic properties are achieved as a result of the polyurethane including hydrophilic polymer blocks, for example polyether groups having a fraction of groups derived from ethylene glycol (CH2CH2O) or from 1,4-butanediol (CH2CH2CH2CH2O) or from 1,3-propanediol (CH2CH2CH2O) or from 1,2-propanediol (—CH(CH3)—CH2O—), or mixtures thereof. Polyetherpolyurethanes may be used as film-forming polymers. The hydrophilic blocks can be constructed in the manner of comb polymers where parts of the side chains or all side chains are hydrophilic polymeric blocks. But the hydrophilic blocks can also be constituents of the main chain (i.e., of the polymer's backbone). One embodiment utilizes polyurethanes where at least the predominant fraction of the hydrophilic polymeric blocks is present in the form of side chains. The side chains can in turn be polyethylene glycol or block copolymers such as poly(ethylene glycol)-co-poly(propylene glycol). If poly(ethylene glycol)-co-poly(propylene glycol) copolymers are used, then the content of ethylene oxide units may be at least 50 mole %, and may be at least 65 mole %.

It is further possible to obtain hydrophilic properties for the polyurethanes through an elevated fraction of ionic groups, carboxylate, sulfonate, phosphonate or ammonium groups. The ammonium groups may be protonated or alkylated tertiary or quarternary groups. Carboxylates, sulfonates, and phosphates may be present as alkali-metal or ammonium salts.

In one embodiment the block copolymers useful herein may be polyether urethanes and polyester urethanes. Polyether urethanes may include polyalkylene glycol units, especially polyethylene glycol units or poly(tetramethylene glycol) units.

As used herein, the term “alkylene glycol” includes both alkylene glycols and substituted alkylene glycols having 2 to 10 carbon atoms, such as ethylene glycol, 1,3-propylene glycol, 1,2-propylene glycol, 1,2-butylene glycol, 1,3-butylene glycol, 1,4-butylene glycol, styrene glycol and the like.

Polyisocyanates may have an average of about two or more isocyanate groups, may have an average of about two to about four isocyanate groups and include aliphatic, cycloaliphatic, araliphatic, and aromatic polyisocyanates, used alone or in mixtures of two or more. Diisocyanates may be used. Aliphatic and cycloaliphatic polyisocyanates, and diisocyanates may be used.

Specific examples of aliphatic diisocyanates include alpha, omega-alkylene diisocyanates having from 5 to 20 carbon atoms, such as 1,6-hexamethylene diisocyanate, 1,12-dodecane diisocyanate, 2,2,4-trimethylhexamethylene diisocyanate, 2,4,4-trimethyl-hexamethylene diisocyanate, 2-methyl-1,5-pentamethylene diisocyanate, and the like. Polyisocyanates having fewer than 5 carbon atoms can be used. Aliphatic polyisocyanates may include 1,6-hexamethylene diisocyanate, 2,2,4-trimethyl-hexamethylene diisocyanate, and 2,4,4-trimethyl-hexamethylene diisocyanate.

Examples of cycloaliphatic diisocyanates may include dicyclohexylmethane diisocyanate, (commercially available as Desmodur® W from Bayer Corporation), isophorone diisocyanate, 1,4-cyclohexane diisocyanate, 1,3-bis(isocyanatomethyl) cyclohexane, and the like. Cycloaliphatic diisocyanates may include dicyclohexylmethane diisocyanate and isophorone diisocyanate.

Specific examples of suitable araliphatic diisocyanates include m-tetramethyl xylylene diisocyanate, p-tetramethyl xylylene diisocyanate, 1,4-xylylene diisocyanate, 1,3-xylylene diisocyanate, and the like. An example araliphatic diisocyanate is tetramethyl xylylene diisocyanate.

Examples of suitable aromatic diisocyanates include 4,4′-diphenylmethane diisocyanate, toluene diisocyanate, their isomers, naphthalene diisocyanate, and the like. An example aromatic diisocyanate is toluene diisocyanate and 4,4′-diphenylmethane diisocyanate.

Examples of high weight average molecular weight compounds a) having 2 or more reactive groups are such as polyester polyols and polyether polyols, as well as polyhydroxy polyester amides, hydroxyl-containing polycaprolactones, hydroxyl-containing acrylic copolymers, hydroxyl-containing epoxides, polyhydroxy polycarbonates, polyhydroxy polyacetals, polyhydroxy polythioethers, polysiloxane polyols, ethoxylated polysiloxane polyols, polybutadiene polyols and hydrogenated polybutadiene polyols, polyacrylate polyols, halogenated polyesters and polyethers, and the like, and mixtures thereof. The polyester polyols, polyether polyols, polycarbonate polyols, polysiloxane polyols, and ethoxylated polysiloxane polyols may be used. Polyesterpolyols, polycarbonate polyols, polyalkylene ether polyols, and polytetrahydrofurane may be used. The number of functional groups in the aforementioned high weight average molecular weight compounds may be on average in the range from 1.8 to 3 and in the range from 2 to 2.2 functional groups per molecule.

The polyester polyols typically are esterification products prepared by the reaction of organic polycarboxylic acids or their anhydrides with a stoichiometric excess of a diol.

The diols used in making the polyester polyols include alkylene glycols, e.g., ethylene glycol, 1,2- and 1,3-propylene glycols, 1,2-, 1,3-, 1,4-, and 2,3-butane diols, hexane diols, neopentyl glycol, 1,6-hexanediol, 1,8-octanediol, and other diols such as bisphenol-A, cyclohexanediol, cyclohexane dimethanol (1,4-bis-hydroxymethylcycohexane), 2-methyl-1,3-propanediol, 2,2,4-trimethyl-1,3-pentanediol, diethylene glycol, triethylene glycol, tetraethylene glycol, polyethylene glycol, dipropylene glycol, polypropylene glycol, dibutylene glycol, polybutylene glycol, dimerate diol, hydroxylated bisphenols, polyether glycols, halogenated diols, and the like, and mixtures thereof. Diols may include ethylene glycol, diethylene glycol, butane diol, hexane diol, and neopentylglycol. Alternatively or in addition, the equivalent mercapto compounds may also be used.

Suitable carboxylic acids used in making the polyester polyols include dicarboxylic acids and tricarboxylic acids and anhydrides, e.g., maleic acid, maleic anhydride, succinic acid, glutaric acid, glutaric anhydride, adipic acid, suberic acid, pimelic acid, azelaic acid, sebacic acid, chlorendic acid, 1,2,4-butane-tricarboxylic acid, phthalic acid, the isomers of phthalic acid, phthalic anhydride, fumaric acid, dimeric fatty acids such as oleic acid, and the like, and mixtures thereof. Polycarboxylic acids used in making the polyester polyols may include aliphatic or aromatic dibasic acids.

Examples of suitable polyester polyols include poly(glycol adipate)s, poly(ethylene terephthalate) polyols, polycaprolactone polyols, orthophthalic polyols, sulfonated and phosphonated polyols, and the like, and mixtures thereof.

An example polyester polyol is a diol. Polyester diols may include poly(butanediol adipate); hexanediol adipic acid and isophthalic acid polyesters such as hexaneadipate isophthalate polyester; hexanediol neopentyl glycol adipic acid polyester diols, e.g., Piothane 67-3000 HNA (Panolam Industries) and Piothane 67-1000 HNA, as well as propylene glycol maleic anhydride adipic acid polyester diols, e.g., Piothane SO-1000 PMA, and hexane diol neopentyl glycol fumaric acid polyester diols, e.g., Piothane 67-SO0 HNF. Other Polyester diols may include Rucoflex® S101.5-3.5, S1040-3.5, and S-1040-110 (Bayer Corporation).

Polyether polyols are obtained in known manner by the reaction of a starting compound that contain reactive hydrogen atoms, such as water or the diols set forth for preparing the polyester polyols, and alkylene glycols or cyclic ethers, such as ethylene glycol, propylene glycol, butylene glycol, styrene glycol, ethylene oxide, propylene oxide, 1,2-butylene oxide, 2,3-butylene oxide, oxetane, tetrahydrofuran, epichlorohydrin, and the like, and mixtures thereof Polyethers may include poly(ethylene glycol), poly(propylene glycol), polytetrahydrofuran, and co [poly(ethylene glycol)-poly(propylene glycol)]. Polyethyleneglycol and polypropyleneglycol can be used as such or as physical blends. In case that propyleneoxide and ethyleneoxide are copolymerized, these polypropylene-co-polyethylene polymers can be used as random polymers or block-copolymers.

In one embodiment, the polyurethane includes side chains, such as poly(alkylene glycol) side chains, sufficient in amount to include about 10 wt. % to 90 wt. %, about 12 wt. % to about 80 wt. %, about 15 wt. % to about 60 wt. %, and about 20 wt. % to about 50 wt. %, of side chain units, e.g. poly(alkylene glycol) units in the final polyurethane on a dry weight basis. The term “final polyurethane” means the polyurethane used for coating the water-absorbing polymeric particles.

The amount of the side-chain units may be (i) at least about 30 wt. % when the weight average molecular weight of the side-chain units is less than about 600 g/mol, (ii) at least about 15 wt. % when the weight average molecular weight of the side-chain units is from about 600 to about 1000 g/mol, and (iii) at least about 12 wt. % when the weight average molecular weight of the side-chain units is more than about 1000 g/mol. Mixtures of active hydrogen-containing compounds having such poly(alkylene glycol) side chains can be used with active hydrogen-containing compounds not having such side chains.

The polyurethanes that may be used may also have reacted therein at least one active hydrogen-containing compound not having the side chains and typically ranging widely in weight average molecular weight from about 50 to about 10000 g/mol, about 200 to about 6000 g/mol, and about 300 to about 3000 g/mol. Suitable active hydrogen-containing compounds not having the side chains include any of the amines and polyols described herein as compounds a) and b).

Suitable immobilization enhancing component herein (which may be applicable from solution) include for example Vector® 4211 (Dexco Polymers, Texas, USA), Vector 4111, Septon 2063 (Septon Company of America, A Kuraray Group Company), Septon 2007, Estane® 58245 (Noveon, Cleveland, USA), Estane 4988, Estane 4986, Estane® X-1007, Estane T5410, Irogran PS370-201 (Huntsman Polyurethanes), Irogran VP 654/5, Pellethane 2103-70A (Dow Chemical Company) and Elastollan® LP 9109 (Elastogran).

Aqueous polyurethane dispersions and polyurethanes that may be used herein are Hauthane HD-4638 (Hauthaway), Hydrolar® HC 269 (COIMolm, Italy), Impraperm® 48180 (Bayer Material Science AG, Germany), Lurapret® DPS (BASF Aktiengesellschaft, Germany), Astacin® Finish LD 1603 (BASF Aktiengesellschaft, Germany), Permax® 120, Permax 200, and Permax 220 (Noveon, Brecksville, Ohio), Syntegra YM2000 and Syntegra YM2100 (Dow, Midland, Mich.), Witcobond® G-213, Witcobond G-506, Witcobond G-507, Witcobond 736 (Uniroyal Chemical, Middlebury, Conn.), Astacin Finish LD 1603, Astacin Finish PUMN TF, Astacin TOP 140, Astacin Finish SUSI (BASF Aktiengesellschaft, Germany) and Impranil® DLF (anionic aliphatic polyester-polyurethane dispersion from Bayer Material Science).

The immobilization enhancing component may include a plasticizer, to facilitate application and coating formation.

### Methods Used Herein:

The following test methods used herein are described in co-pending application WO2006/083585, i.e. methods for: preparation of films of the elastic film-forming polymer; polymer molecular weights determination; water-swelling capacity of a polymer determination.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent structure comprising:
a substrate material; and
an absorbent component, the absorbent component comprising:
a matrix material;
water-absorbing polymeric particles; and
an immobilization enhancing component applied to a surface of the water-absorbing polymeric particles to form a coating or partial coating thereon and to form an area of attachment for the matrix material, to aid immobilization of the water-absorbing polymeric particles by the matrix material in the absorbent structure.

2. The absorbent structure of claim 1, wherein the matrix material comprises:
a thermoplastic adhesive material, having a viscosity of from about 1600 to about 2500 mPa·s at 175° C., as determined by the method set out herein; and
a thermoplastic adhesive material, having a viscosity of from about 2300 to about 3700 mPa·s, at 149° C., as determined by the method set out herein

3. The absorbent structure of claim 1, wherein the immobilization enhancing component comprises a polymeric material having a polyolefin, polyester, polyether, polyamide, poly urea and polyurethane units; and wherein the matrix material comprises a polymeric material having polyolefin, polyester, polyether, polyamide units, poly urea and polyurethane units; and wherein the immobilization enhancing component is applied to the water-absorbing polymeric particles to form a coating or partial coating prior to addition of the matrix material to the coated or partially coated water-absorbing polymer material.

4. The absorbent structure of claim 3, wherein the immobilization enhancing component comprises a film-forming copolymeric or block copolymeric material; wherein the matrix material comprises a thermoplastic adhesive component; and wherein the immobilization enhancing component includes a first solubility parameter and the matrix component or thermoplastic adhesive component thereof, includes a second solubility parameter, and wherein the difference between the first solubility parameter and the second solubility parameter is from 0 to 1 (cal/cm3)½.

5. The absorbent structure of claim 1, wherein the water-absorbing polymeric particles comprising a coating or partial coating of the immobilization enhancing component, have a mean particle size of from 200 microns to 800 microns, whereby no more than 10% by weight of the particles have a particle size of less than 150 microns.

6. The absorbent structure of claim 1, wherein the immobilization enhancing component is present at a level of 0.01% to 2%, or up to 1% by weight of the absorbent component, and wherein a weight ratio of the water-absorbing polymers to the matrix material is from about 35:1 to about 100:1.

7. The absorbent structure of claim 1, wherein the matrix material is a fibrous, adhesive material, comprising an ethylene vinyl acetate derivative, styrenic block copolymer derivative, and a polyolefin derivative; and wherein the immobilisation enhancing component comprises polyurethane polymers.

8. The absorbent structure of claim 6, comprising less than about 5% by weight of absorbent cellulose material, and from about 1% to about 10% by weight of the matrix material.

9. An absorbent structure comprising:
a substrate material; and
an absorbent component comprising
(a) water-absorbing polymeric particles, the particles having a coating or partial coating of an immobilization enhancing component; and
(b) a matrix material, whereby the weight ratio of the component (a) to (b) is from 10:1 to 100:1 and the immobilization enhancing component is present at a weight level (by weight of the absorbent component) of from 0.01% to 1% by weight, and whereby the water-absorbing polymeric particles with the coating or partial coating have a mean particle size of from 200 to 800 microns and a particle size distribution such that less than 10% by weight of the particles has a particle size of below 150 microns.

10. The absorbent structure of claim 9, comprising less than about 5% by weight of absorbent cellulose material, and from about 1% to about 10% by weight of the matrix material.
